(19) United States
(12) Patent Application Publication
(10) Pub. No.: US 2014/0000361 A1
(43) Pub. Date: Jan. 2, 2014

(54) SENSOR APPARATUS FOR DETERMINING FORCES APPLIED TO A PEDAL OF A BICYCLE

(71) Applicant: SPECIALIZED BICYCLE COMPONENTS, INC., Morgan Hill, CA (US)

(72) Inventor: Chuck Teixeira, Bonny Doon, CA (US)

(73) Assignee: SPECIALIZED BICYCLE COMPONENTS, INC., Morgan Hill, CA (US)

(21) Appl. No.: 13/664,575

(22) Filed: Oct. 31, 2012

Related U.S. Application Data
(63) Continuation-in-part of application No. 13/535,978, filed on Jun. 28, 2012.

(51) Int. Cl. A61B 5/22

(52) U.S. Cl. 73/379.01

(57) ABSTRACT

A bicycle including a frame that has a bottom bracket and a crankset attached to the bottom bracket. The crankset includes a sprocket assembly and a spider that has a flexible arm coupled to the sprocket assembly. The bicycle also includes a pedal coupled to the crankset and operable to propel the bicycle in response to a force acting on the pedal, and a sensor apparatus. The sensor apparatus has a sensor element positioned to sense a force transferred from the pedal to the sprocket assembly and indicative of the force acting on the pedal.

### BACKGROUND

The present invention relates to bicycles, and more particularly to a bicycle including a sensor apparatus for measuring forces applied to the pedals of the bicycle.

Typically, bicycles are propelled by pedals mounted to a crankset at opposite ends of a spindle. A typical crankset is equipped with two cranks that each supports a pedal at one end and couples with a spindle adjacent the other end. These cranksets transfer energy exerted on the pedals by a rider to forward motion of the bicycle. The crankset typically includes one or more sprockets that engage a chain to transfer the rotary motion of the crankset to a rear wheel.

Often, it is desirable to know the directional forces applied to the pedals by a rider so that the power associated with the rider can be accurately determined. Some existing bicycles include power meters located at the rear hub of the bicycle. Other systems determine the power of the rider using sensors that are inserted into the pedal or the crank arm. Such systems typically require custom-made components to accommodate the power meters.

### SUMMARY

In one construction, the present invention provides a bicycle including a frame that has a bottom bracket and a crankset attached to the bottom bracket. The crankset includes a sprocket assembly and a spider that has a flexible arm coupled to the sprocket assembly. The bicycle also includes a pedal coupled to the crankset and operable to propel the bicycle in response to a force acting on the pedal, and a sensor apparatus. The sensor apparatus includes a sensor element positioned to sense a force transferred from the pedal to the sprocket assembly and indicative of the force acting on the pedal.

In another construction, the present invention provides a bicycle including a frame that has a bottom bracket, and a crankset attached to the bottom bracket and including a sprocket assembly and a spider. The spider has a first spider element and a second spider element separated from the first spider element. The first spider element is movable relative to the second spider element. The bicycle also includes a pedal coupled to the crankset and operable to propel the bicycle in response to a force acting on the pedal, and sensor apparatus coupled to the spider. The sensor apparatus includes a sensor element disposed between the first spider element and second spider element to sense a force transferred from the pedal to the sprocket assembly and indicative of the force acting on the pedal.

In another construction, the present invention provides a crankset for a bicycle including a pedal. The crankset includes a sprocket assembly, a spider including a central portion and an arm, and a sensor apparatus. The spider is rotatable in response to a force applied to the pedal. The arm is disposed between the sprocket assembly and the central portion and is flexible in response to rotation of the central portion. The sensor apparatus includes a sensor element positioned to sense the force applied to the pedal in response to yielding of the flexible arm.

Other aspects of the invention will become apparent by consideration of the detailed description and accompanying drawings.

### BRIEF DESCRIPTION OF THE DRAWINGS

FIG. 1 is a side view of a bicycle including a crankset and sensor apparatus embodying the present invention.

FIG. 2 is a perspective view of a crank arm of the crankset, the sensor apparatus, and a pedal.

FIG. 3 is an exploded view of the assembly of FIG. 2.

FIG. 4 is an enlarged view of a portion of the assembly of FIG. 3.

FIG. 5 is a side view illustrating the crank arm, the sensor apparatus, and the pedal in three rotational positions and associated vector forces applied to the pedal.

FIG. 6 is a section view of the crank arm, the sensor apparatus, and the pedal taken along line 6-6 in FIG. 2.

FIG. 7 is a section view similar to FIG. 6 illustrating the crank arm, the sensor apparatus, and the pedal when pressure is applied to the pedal by a rider.

FIG. 8 is a section view of the crank arm, the sensor apparatus, and the pedal taken along line 8-8 in FIG. 2.

FIG. 9 is a schematic view illustrating a portion of an electrical circuit of the sensor apparatus.

FIG. 10 is an enlarged section view of another housing for the sensor apparatus of FIG. 2.

FIG. 11 is a section view of a housing for another sensor apparatus embodying the invention.

FIG. 12 is a section view of the sensor apparatus of FIG. 11 illustrating a force acting on the housing.

FIG. 13 is a perspective view of the sensor apparatus of FIG. 11 coupled to a spider of the bicycle of FIG. 1.

FIG. 14 is an exploded perspective view of the sensor apparatus and the spider of FIG. 13.

FIG. 15 is a perspective view of a portion of the spider of FIG. 13.

FIG. 16 is a section view of the spider and the sensor apparatus of FIG. 13.

FIG. 17 is a perspective view of another spider and sensor apparatus embodying the present invention.

FIG. 18 is an exploded perspective view of the spider and sensor apparatus of FIG. 17 with chain rings removed.

FIG. 19 is a side view illustrating the spider of FIG. 18 with cover plates removed and the sensor apparatus coupled to the spider.

FIG. 20 is a side view illustrating the spider of FIG. 17 with cover plates removed and the sensor apparatus coupled to the spider.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

### DETAILED DESCRIPTION

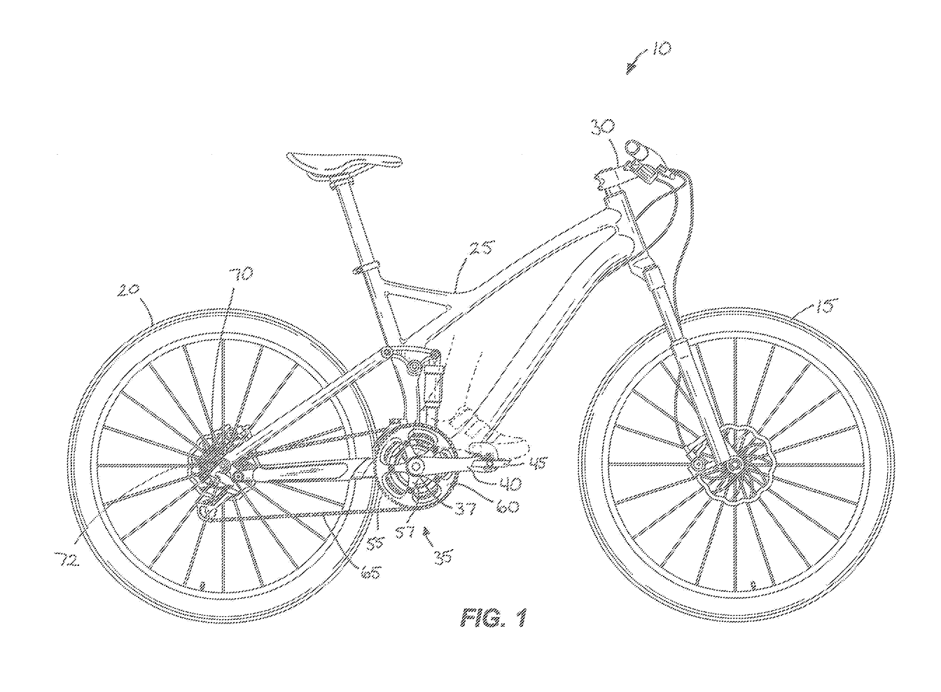

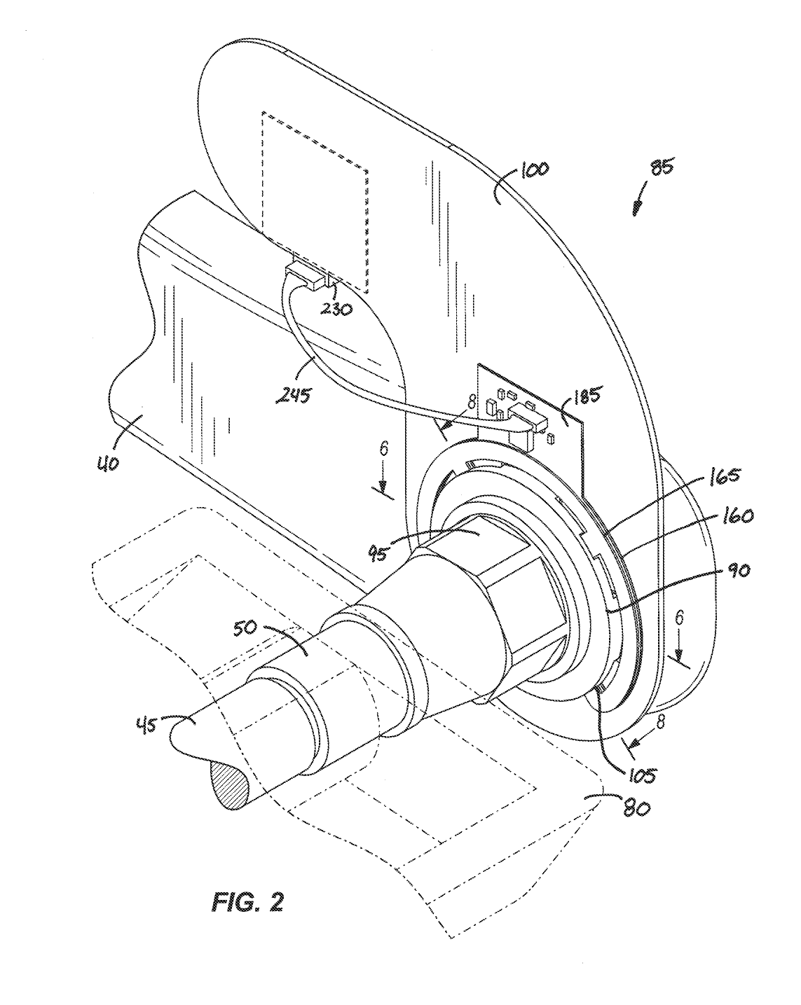

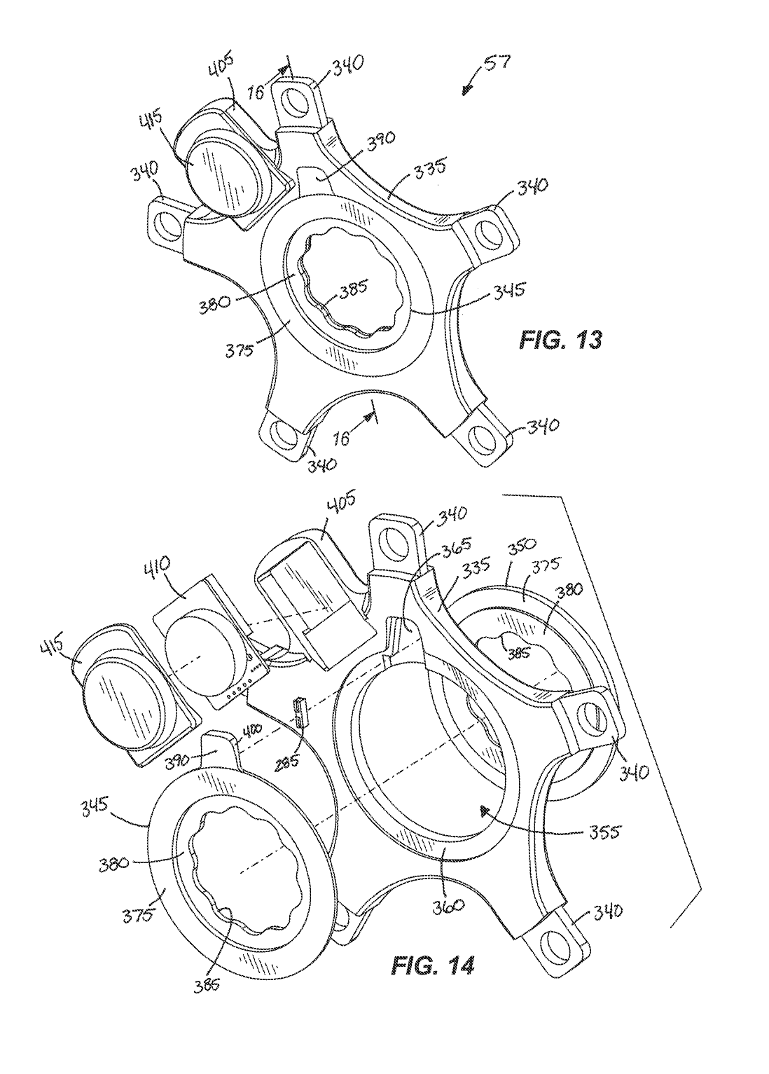

FIG. 1 illustrates a bicycle 10 that includes a front wheel 15, a rear wheel 20, a frame 25, a steering assembly 30, and a crankset 35 attached to a bottom bracket 37 of the frame 25. With reference to FIGS. 1 and 2, the crankset 35 includes opposed crank arms 40 (one shown) and pedals 45 (one shown) that are attached to distal ends of the crank arms 40 via pedal spindles 50 to allow a rider to rotate the crankset 35 and to propel the bicycle 10, as is known in the art. A front sprocket assembly 55 is coupled to the crankset 35 by a spider 57 (FIGS. 1 and 13) and may include one or more chain rings 60 that couple to a chain 65. The chain 65 engages the rear wheel 20 through a series of rear sprockets 70 connected to a rear hub 72.

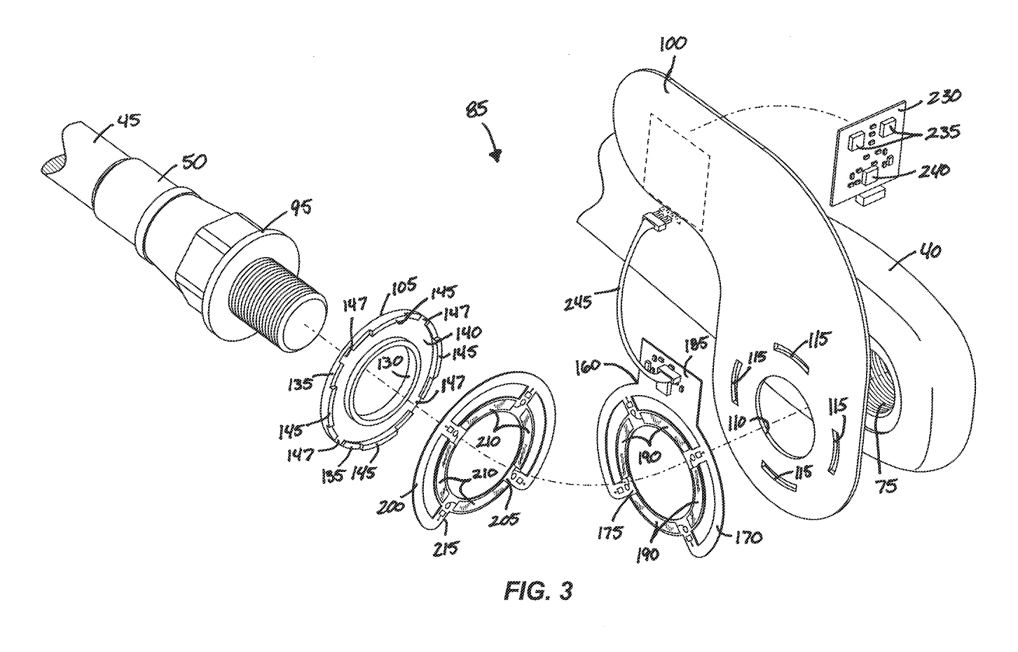

FIGS. 2 and 3 show that each pedal spindle 50 has a shaft that is inserted (e.g., threaded) into a hole 75 in the distal end of the crank arm 40. The pedal 45 includes a cage 80 (e.g., for a clipless pedal 45) that rotates about the pedal spindle 50 so that pedal 45 can move with the rider's foot.

FIGS. 2, 3, and 5-8 illustrate a sensor apparatus or capsule 85 positioned between the crank arm 40 and the pedal 45 (e.g., around the shaft of the pedal spindle 50) to determine the directional (i.e., vector) forces and the corresponding power applied by the rider to the pedal 45. While the sensor apparatus 85 is described below with regard to a capacitive sensor, other sensors (e.g., a piezo sensor, an optical sensor, a pressure sensor, a strain gauge sensor such as a wavy plate strain sensor, etc.) can be placed between the crank arm 40 and the pedal 45 to determine the directional forces applied to the pedal 45.

In particular, the sensor apparatus 85 is received on an end of the pedal spindle 50 and includes a housing 90 that is located axially along the pedal spindle 50 between the crank arm 40 and a fastener head or flange 95 of the pedal spindle 50 that is proximate the crank arm 40. Stated another way, the sensor apparatus 85 is sandwiched between the crank arm 40 and the flange 95 (e.g., abutting the crank arm 40 and the flange 95) so that the sensor apparatus 85 is held in engagement with the crank arm 40 when the pedal spindle 50 is inserted and tightened on the crank arm 40 via the flange 95. As illustrated, a washer 97 is disposed in a recess 98 of the crank arm 40 and surrounds the pedal spindle 50.

With reference to FIGS. 2, 3, 6, and 7, the illustrated housing 90 includes a back plate 100 that abuts the crank arm 40 and a shell 105 that is coupled to the back plate 100 and that abuts the pedal spindle 50. The back plate 100 is a flat plate that holds the washer 97 in the recess 98 of the crank arm 40, and that defines a first side or wall of the housing 90. The back plate 100 is attached (e.g., adhered, formed integrally with, etc.) to the crank arm 40 to provide a rigid mounting surface for the shell 105. As illustrated, the back plate 100 is elbow shaped, and has a first hole 110 for allowing passage of the pedal spindle, and circumferentially-spaced apertures 115 surrounding the first hole 110. As will be appreciated, the back plate 100 can have other shapes and can be integrally formed with the crank arm 40.

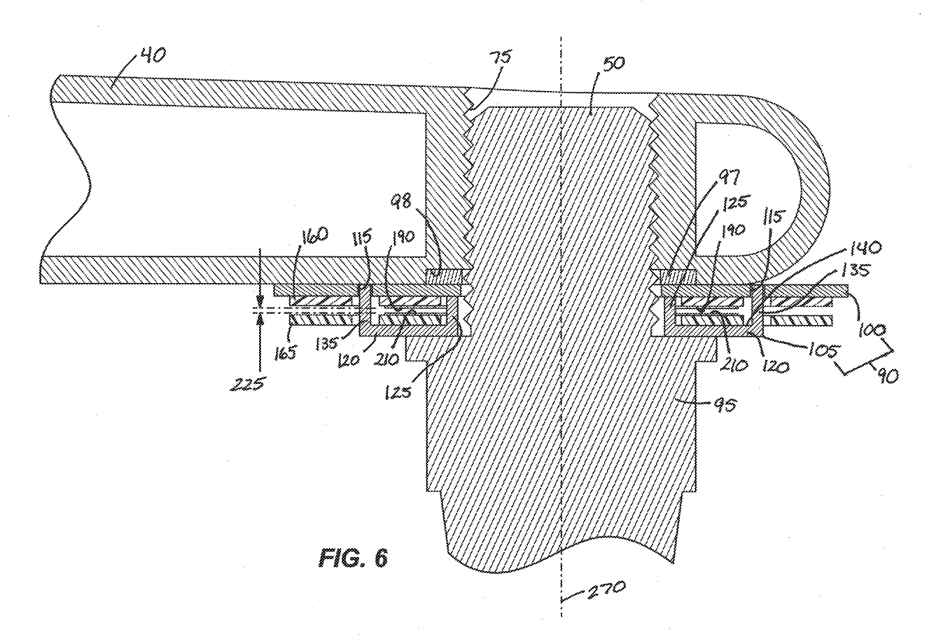

The illustrated shell 105 is defined by an open-ended doughnut-shaped structure that has an outer wall 120, an inner radial wall 125 extending from the outer wall 120 and defining a second hole 130, and an outer radial wall 135 extending from the outer wall 120. The outer radial wall 135 is spaced from the inner radial wall 125 to form a hollow area or trough 140. With reference to FIGS. 3 and 6, the outer radial wall 135 defines tabs 145 that extend farther from the outer wall 120 than the most distal part of the inner radial wall 125. The tabs 145 align and couple to the back plate 100 within the apertures 115 so that the trough 140 is fully or substantially enclosed by the remaining portions of the outer radial wall 135, and so that the outer wall 120 does not move relative to the back plate 100. With the exception of small recesses 147, which are further explained below, the portions of the outer radial wall 135 between the tabs 145 abut the back plate 100 when the shell 105 is attached to the back plate 100.

FIG. 10 illustrates an alternative housing 150 for the sensor apparatus 85. The only difference between the housing 90 described with regard to FIGS. 2, 3, 5, 6, and 7 and the housing 150 illustrated in FIG. 10 is that the shell 105 of the housing 150 has an annular rib 155 on the inside of the outer radial wall 135 that forms a shelf. The shelf engages or abuts the back plate 100 continuously around the shell 105 to provide additional rigidity to the shell 105 relative to the back plate 100.

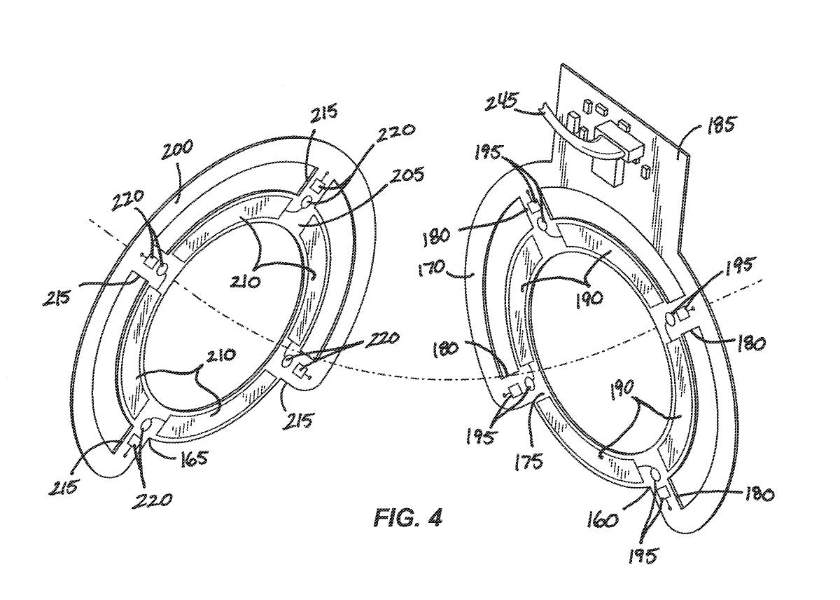

With reference to FIGS. 2-4, 6, and 7, the sensor apparatus 85 also includes a first sensor element 160 in the form of a first substrate (e.g., a printed circuit board), and a second sensor element 165 in the form of a second substrate (e.g., a printed circuit board). The first sensor element 160 is mounted to the crank arm 40 via the back plate 100 (e.g., adhered to the back plate 100). FIGS. 3 and 4 show that the first sensor element 160 is defined by a first radial arm 170 and a first concentric platform 175 that is connected to the radial arm by first bridges 180. The first radial arm 170 has an enlarged area that supports a first detector or sensor board 185.

As shown in FIGS. 3 and 4, the first platform 175 supports first sensors 190 that are circumferentially spaced from each other and that are in electrical communication with the sensor board 185 via electrical contact points 195 on the first bridges 180. The first bridges 180 are relatively small compared with the size of the first sensors 190 to maximize the sensing capacity of the first sensor element 160. As illustrated, the first sensor element 160 includes four circumferentially-spaced sensors and four corresponding bridges and contact points 195, although fewer or more than four sensors and corresponding bridges and contact points 195 are possible and considered herein. The sensors 190 are in electrical communication with the contact points 195 via circuit board material that is printed on the first sensor element 160.

Referring to FIGS. 3, 4, 6, and 7, the second sensor element 165 is mounted (e.g., adhered) to the outer wall 120 of the shell 105 within the trough 140 so that the second sensor element 165 is positioned adjacent the pedal 45. FIGS. 3 and 4 show that the second sensor element 165 is defined by a second radial arm 200 and a second concentric platform 205 that is connected to the first radial arm 170 by second bridges 215. With the exception of the sensor board 185 on the first sensor element 160, the first sensor element 160 and the second sensor element 165 are a mirror image of each other. In particular, the second sensor element 165 has a substantially circular cross-sectional shape that includes four circumferentially-spaced second sensors 210. The second sensors 210 are in electrical communication with the sensor board 185 via respective electrical contact points 220 on the second bridges 215. More specifically, the sensors 210 are in electrical communication with the contact points 220 via circuit board material that is printed on the second sensor element 165. With reference to FIGS. 4 and 8, the contact points 220 of the second sensor element 165 are soldered to the contact points 195 of the first sensor element 160 so that signals from the second sensors 210 can be transmitted to the first sensor board 185 through the circuit board material of the first sensor element 160.

With reference to FIG. 6, when the sensor apparatus 85 is assembled, the second sensor element 165 is spaced apart from the first sensor element 160 to define a relatively small gap 225 (e.g., 0.1 mm-0.3 mm). The gap 225 can be filled with any suitable compressible medium (e.g., gas such as air, a resin, a thin strip of material such as tape, etc.). In addition, the second sensors 210 are aligned with and face the first sensors 190. The first and second sensors 190, 210 are complementary to each other and determine the size of the gap 225, or conversely, the thickness of the compressible medium. The illustrated first and second sensors 190, 210 are capacitor plates that cooperatively determine the gap size (or the thickness of the compressible medium), although other sensors (e.g., piezo sensors, pressure sensors, strain gauge sensors, etc.) are possible.

With continued reference to FIGS. 2-4, the sensor apparatus 85 further includes a second detector or sensor board 230 that is located remotely from the first sensor board 185. As illustrated, the second sensor board 230 is attached to the back plate 100 (e.g., on the opposite side). However, the second sensor board 230 can be attached to the crank arm 40 in any suitable location. The second sensor board 230 includes an accelerometer 235 for determining the magnitude and direction of acceleration of the pedal 45, and a transmitter 240 that can communicate with a remote device (e.g., display, data logger, battery, etc.). The second sensor board 230 is electrically connected to the first sensor board 185 via a wire 245, although other connections (e.g., wireless) are possible.

The sensor apparatus 85 is assembled by attaching the first sensor element 160 to the back plate 100 and attaching the second sensor element 165 to the shell 105. The shell 105 is then attached to the back plate 100 by engagement of the tabs 145 with the apertures 115. As will be appreciated, the back plate 100 and shell 105 can be permanently joined together (e.g., welded, adhered, etc.) after the first and second sensor elements 160, 165 are put in place. In the assembled state, the bridges 180, 215 extend through the outer radial wall 135 through the recesses 147 to provide communication from within the housing 90 to the sensor board 185. The assembled sensor apparatus 85 is coupled to the bicycle 10 by inserting the pedal spindle 50 through the first and second holes 110, 130 of the housing 90, and then attaching (e.g., threading) the pedal spindle 50 to the crank arm 40.

The pedal spindle 50 is attached to the crank arm 40 with a predetermined amount of force (e.g., 28, N-m). In this manner, the amount of pre-stress on the sensor apparatus 85 is known. Knowing the pre-stress, the sensor apparatus 85 has a baseline measurement for the size of the gap 225 (or material thickness) so that a change relative to the baseline measurement can be determined. Generally, the sensor apparatus 85 determines the vector forces applied to the pedal 45 when the rider engages the pedal 45 to move the bicycle 10 forward as well as the tangential velocity of the pedal 45, which is determined using the accelerometer 235. In particular, the sensor apparatus 85 determines the tangential force and the radial force applied to the pedal 45 and determines the overall power of the rider based on the amount and direction of the forces and the tangential pedal velocity.

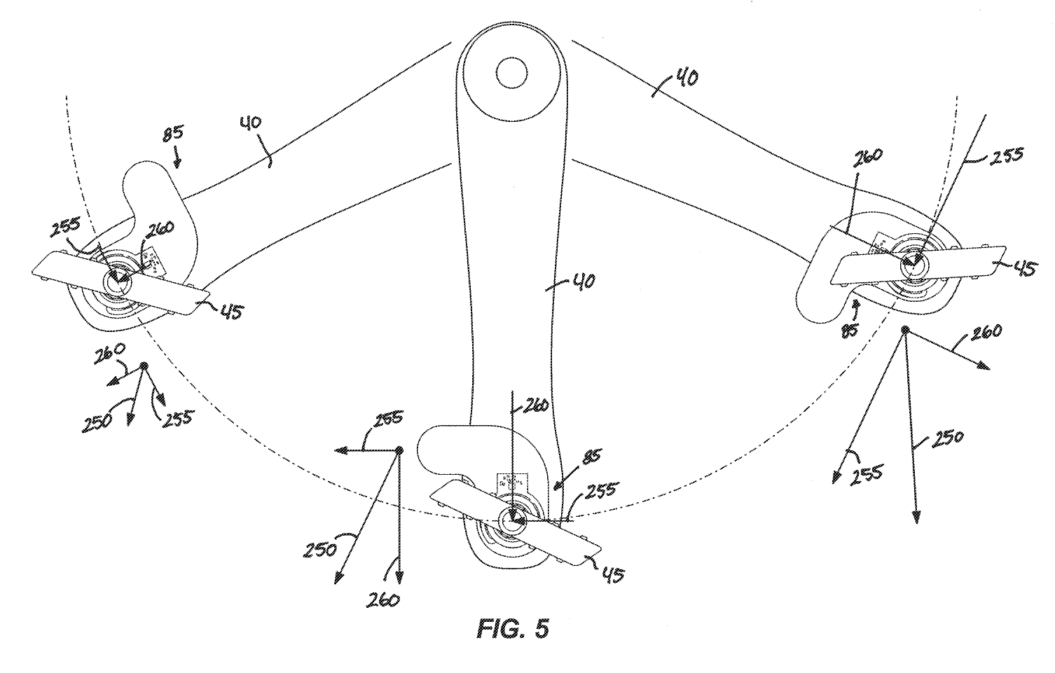

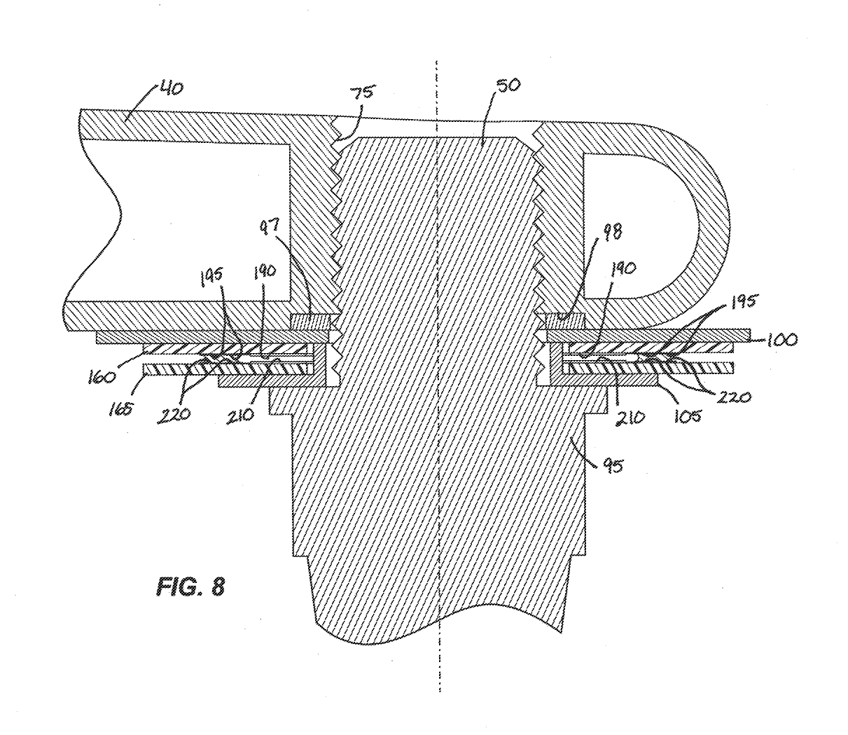

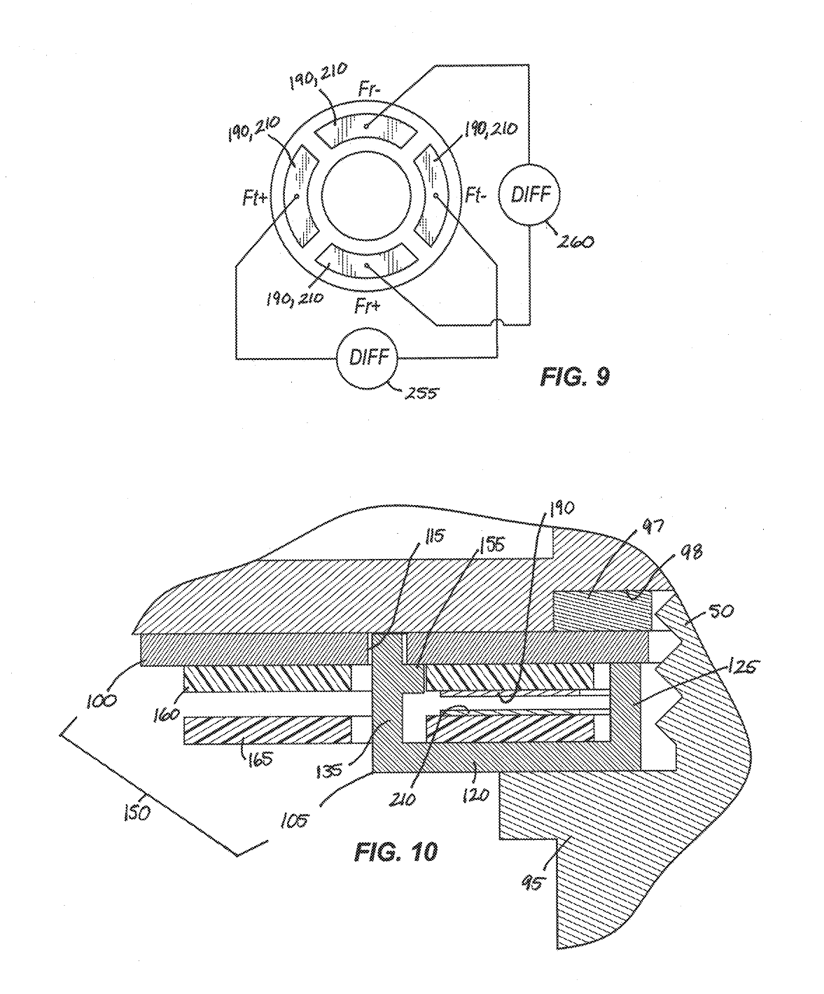

Referring to FIGS. 5-9, when a rider pushes or pulls on the pedal 45 (depending on the radial orientation of the pedal 45 relative to the bicycle 10), the force vector 250 associated with the rider's engagement of the pedal 45 has a useful tangential force vector 255 along the arcuate path of the pedal 45 and a radial force vector 260 (unusable or wasted force) in a direction along the crank arm 40. The amount of tangential and radial force vectors 255, 260 are determined by the sensor apparatus 85 based on a change in size of the gap 225 between pairs of opposing sensors 190, 210.

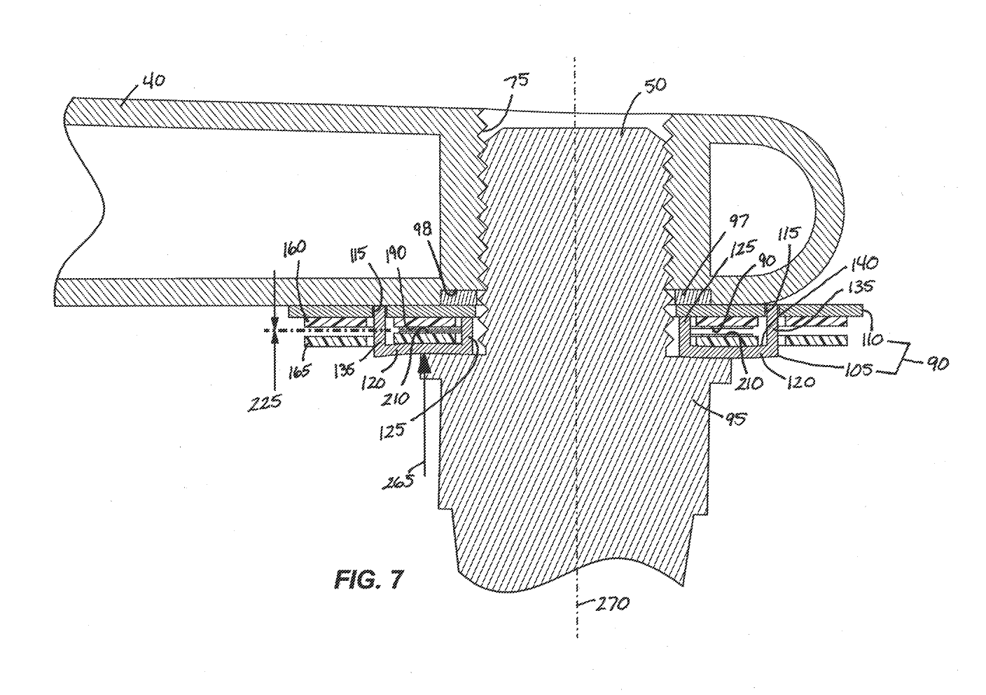

When pressure is applied to the pedal 45, the resulting force is transferred from the pedal spindle 50 to the crank arm 40 by the shaft. As shown in FIG. 7, the force (indicated by arrow 265) deflects the pedal spindle 50 a small amount, which in turn deforms the outer wall 120 of the shell 105. For comparison, FIG. 6 shows the pedal 45 without pressure from the rider (a non-deformed state). Generally, a substantial portion of the force acting on the pedal 45 is transferred directly through the pedal spindle 50 to the crank arm 40. Only a small portion of the force acts on the sensor apparatus 85. Stated another way, the pedal spindle 50 is directly acted upon by the pedal 45 in response to pressure applied to the pedal 45, and transfers most of the force directly to the crank arm 40.

Deflection of the pedal spindle 50 (e.g., generally longitudinally inward along the crank arm 40 as shown in FIG. 7) causes a portion of the outer shell 105 and the second sensor element 165 (the left side of the housing 90 in FIG. 7) to move toward the first sensor element 160 a small amount in a direction parallel to a pedal axis 270, while also causing the opposed portion of the outer shell 105 and the second sensor element 165 (the right side of the housing 90 in FIG. 7) to move away from the first sensor element 160 a small amount in the opposite direction. The change in the gap 225 on both sides of the housing 90 is detected by the sensor apparatus 85, and the difference is used to determine the corresponding tangential and radial forces 255, 260 being applied to the pedal 45.

In particular, the sensor board 185 senses the force transferred from the pedal spindle 50 to the crank arm 40 via detecting the change in distance or change in volume between the first sensor element 160 and the second sensor element 165 using all four sensors 190. The sensor board 185 determines the amount of the directional forces 255, 260 that are being applied to the pedal 45 based on the change in distance or change in volume. With reference to FIG. 8, two opposed sensors 190, 210 of the first and second sensor elements 160, 165 cooperatively determine the tangential force 255 and the remaining two opposed sensors 190, 210 of the first and second sensor elements 160, 165 determine the radial force 260 based on the change in size of the gap 225 between the respective sensors 190, 210. These directional forces 255, 260 are then communicated to the second sensor board 230, which determines the tangential velocity of the pedal 45 and the corresponding power of the rider in part using the accelerometer 235. This information can then transferred to the remote device (not shown).

The sensor apparatus 85 provides a separate sensor component that can be used universally with existing crank arms 40 and pedals 45 without much, if any, modification of the crank arms 40 and the pedals 45. The sensor apparatus 85 can be attached to one or both sides of the bicycle 10 so that the directional forces associated with pressure on the pedal 45 can be determined for the rider's left and/or right leg.

Placement of the sensor apparatus 85 between the crank arm 40 and the pedal spindle 50, which is acted upon directly by the pedal 45, provides accurate measurements of the resultant force vector 250 stemming from the force applied to the pedal 45. Stated another way, by sandwiching the sensor apparatus 85 between the crank arm 40 and the pedal 45, accurate measurements can be taken of the directional forces 255, 260 and acceleration (i.e., the position and tangential velocity of the pedal 45) resulting from pressure applied to the pedal 45 so that the power of the rider can be determined. Furthermore, the sensor apparatus 85 is located so that force applied to the pedal 45 directly acts on the sensor elements 160, 165. As a result, separate (i.e., independent) and accurate measurements of the power generated by the rider's left and right legs can provide valuable data that can be used to evaluate and improve the rider's ability.

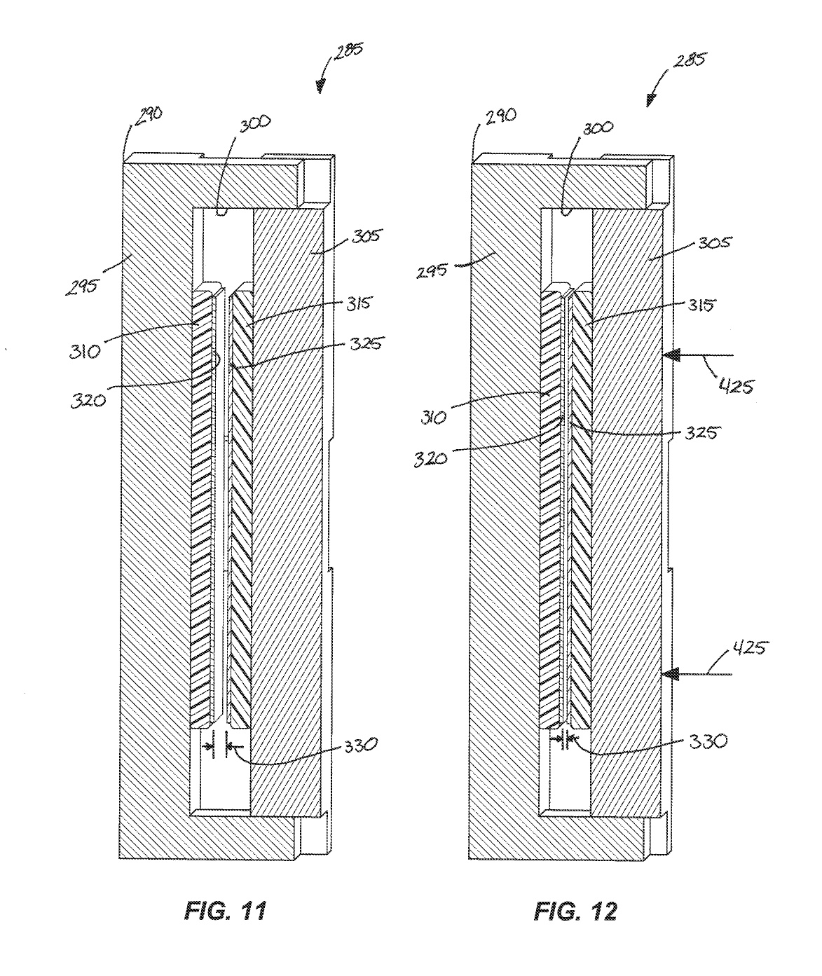

FIGS. 11 and 12 illustrate another sensor apparatus or capsule 285 that can be positioned on the bicycle 10 in lieu of or in addition to the sensor apparatus 85 to determine the force applied by the rider to the pedal 45. For example, the sensor apparatus 285 can be located in the spider 57 (see FIG. 13), although the sensor apparatus 285 can be positioned in other locations (e.g., the bottom bracket 37, the rear hub 72, etc.). Except as described below, the sensor apparatus 285 is the same as the sensor apparatus 85 described with regard to FIGS. 2-10.

The illustrated sensor apparatus 285 includes a housing 290 that has a cup-like back plate or shell 295 defining a hollow area or trough 300, and a cap plate 305 engaged with the back plate 295 (e.g., via flexible material so that the cap plate 305 can move relative to the shell 295) to enclose the trough 300. Alternatively, either or both the back plate 295 and the cap plate 305 can be cup-like in shape. Generally, the structure of the housing 290 can vary based on where the sensor apparatus 285 is located on the bicycle 10. Also, the shape of the housing 290 can be modified to fit the location on the bicycle 10.

With continued reference to FIGS. 11 and 12, the sensor apparatus 285 also includes a first sensor element 310 in the form of a first substrate (e.g., a printed circuit board), and a second sensor element 315 in the form of a second substrate (e.g., a printed circuit board). The illustrated first sensor element 310 and the second sensor element 315 are a mirror image of each other. The first sensor element 310 is mounted to (e.g., adhered to) the back plate 295 and supports a first sensor 320. The second sensor element 315 is mounted to (e.g., adhered to) the cap plate 305 and supports a second sensor 325. The sensors 320, 325 are in electrical communication with a sensor or detector board (not shown) via circuit board material printed on the first and second sensor elements 310, 315. Unlike the sensor apparatus 85, the sensor apparatus 285 has only one first sensor 320 and one second sensor 325. Stated another way, the sensor apparatus 285 incorporates only one quadrant of the sensor apparatus 85 into the housing 290.

The sensor apparatus 285 is assembled by attaching the first sensor element 310 to the back plate 295 and attaching the second sensor element 315 to the cap plate 305. The cap plate 305 is then attached to the back plate 295. The assembled sensor apparatus 285 is then coupled to the bicycle 10.

When the sensor apparatus 285 is assembled, the second sensor element 315 is spaced apart from the first sensor element 310 to define a relatively small gap 330 (e.g., 0.1 mm-0.3 mm) that can be filled with any suitable compressible medium (e.g., gas such as air, a resin, a thin strip of material such as tape, etc.). Also, the second sensor 325 is aligned with and faces the first sensor 320. The first and second sensors 320, 325 (e.g., capacitive sensors, strain gauges, piezo sensors, pressure sensors) are complementary to each other and determine the size of the gap 330, or conversely, the thickness of the compressible medium.

FIGS. 13-16 show that the sensor apparatus 285 (one shown) is disposed in the spider 57 to detect the force applied by the rider to the pedals 45. With reference to FIGS. 13 and 14, the spider 57 has a central body or central portion 335, arms 340 radially extending outward from the central portion 335, a first insert 345 coupled to the central portion 335, and a second insert 350 coupled to the central portion opposite the first insert 345. The arms 340 attach the front sprocket assembly 55 to the central portion 335.

The central portion 335 has a hollow 355 located at the center of the spider 57. On both sides (one shown) of the spider 57, the central portion 335 has a recessed inner periphery 360 that surround the hollow 355. As shown in FIGS. 14 and 16, the central portion 335 also has a cavity 365 that is radially offset from the center of the spider 57 and that is located radially in-line with and extending partially along one arm 340. The cavity 365 is in communication with the hollow 355 and extends deeper into the side of the spider 57 than the recessed inner periphery 360. With reference to FIG. 16, the shell 295 of the sensor apparatus 285 is attached to a sidewall 370 that partially defines the cavity 365. Although the illustrated spider 57 has one sensor apparatus 285 positioned in the cavity 365, the spider 57 can include several sensor apparatuses 285 (e.g., one for each arm 340).

With reference to FIGS. 13-16, each of the first insert 345 and the second insert 350 has a rim 375 coupled to the spider 57 and a spindle portion 380 extending radially inward from and around the rim 375. Each rim 375 is engaged with the central portion 335 within the respective recessed inner periphery 360 so that the inserts 345, 350 are nested in the spider 57. The spindle portions 380 partially overlap or cover the hollow 355 and have respective apertures 385 that is sized and shaped to fit onto a spindle (not shown) of the crankset 35.

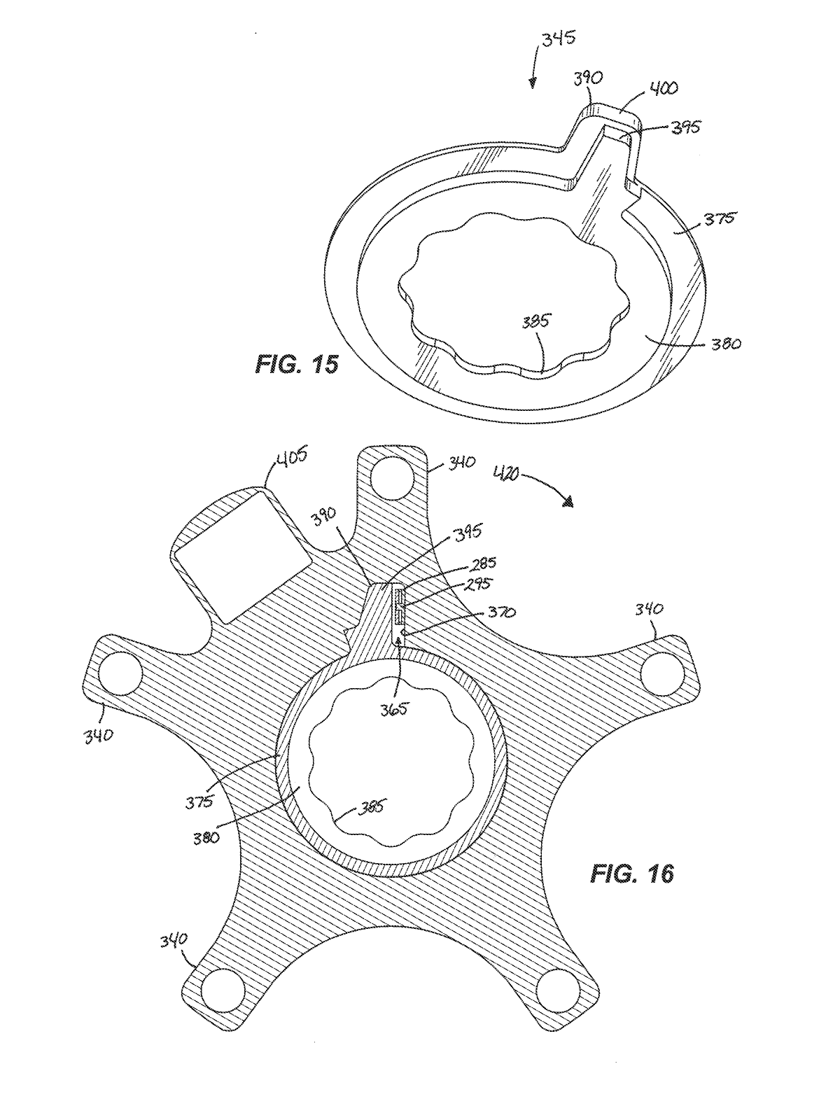

FIGS. 13-16 show that the first insert 345 also has a spider engagement 390 extending radially outward from the rim 375 and recessed in the spider 57. As illustrated, the spider engagement 390 has a first portion 395 that is disposed in the cavity 365, and a second portion 400 that overlays the cavity 365 to cover the sensor apparatus 285. As shown in FIGS. 15 and 16, the first portion 395 is shaped to generally conform to the shape of the cavity 365 and is sized to be smaller than the cavity 365 to accommodate the sensor apparatus 285. The sensor apparatus 285 is positioned between the central portion 335 and the spider engagement 390 so that the first and second sensor elements 310, 315 are responsive to a force transferred from the first insert 345 to the central portion 335 to detect the vector force acting on the pedal. In other words, the spider engagement 390 is operatively coupled to the central portion 335 through the sensor apparatus 285 to transfer a force from the crank arm 40 to the spider 57 (i.e., between the first insert 345 and the central portion 335). In some constructions, the first sensor element 310 can be directly coupled to the spider engagement 390 and the second sensor element 315 can be coupled to a wall of the cavity 365 without the housing 290 to determine the force transferred between the crank arm 40 and the spider 57.

The first insert 345 is rotatable relative to the central portion 335 so that the sensor apparatus 285 can detect the force being transferred from the crank arm 40 to the spider 57. As illustrated, the first portion 395 is spaced a small distance (e.g., less than 1 mm) from the sensor apparatus 285 absent a force on the pedal 45, although the first portion 295 can rest against the sensor apparatus 285. As shown in FIGS. 13-15, the second portion 400 is sized to completely enclose the cavity 365.

With continued reference to FIGS. 13 and 14 and 16, the spider 57 also includes a housing 405 that is attached to the central portion 335 between two arms 340. An electronic module 410 is disposed in the housing 405 and is enclosed by a cover 415. The electronic module 410 is in communication with the sensor apparatus 285 (e.g., by wired or wireless connection) to detect the change in the gap 330 between the first sensor element 310 and the second sensor element 315 and thus determine the force being applied to the pedal 45 by the rider. As shown, the electronic module 410 is located adjacent the sensor apparatus 285 and has a power source (e.g., a battery) to provide power for the sensor apparatus 285 and for communicating data to a remote location (e.g., a computer mounted on the bicycle 10).

The sensor apparatus 285 determines the absolute force 250 that is applied to the pedal 45 when the rider engages the pedal 45 to move the bicycle 10 forward. As discussed with regard to FIG. 5, when the rider pushes or pulls on the pedal 45 (depending on the radial orientation of the pedal 45 relative to the bicycle 10), the force vector 250 associated with the rider's engagement of the pedal 45 has a useful tangential force vector 255 along the arcuate path of the pedal 45 and a radial force vector 260 (unusable or wasted force) in a direction along the crank arm 40. Because the sensor apparatus 285 only has one each of the first sensor 320 and the second sensor 325 (i.e., the sensor apparatus 285 does not have multiple quadrants of sensors) only the magnitude of the force vector 250 is determined by the sensor apparatus 285 based on a change in size of the gap 330 between the opposing sensors 320, 325.

The first insert 345 is coupled to the central portion 335 so that the insert 345 can move (i.e., rotate) a small amount relative to the spider 57. The inserts 345, 350 are positioned between the bottom bracket 37 and the crank arm 40 so that the inserts 345, 350 are held in lateral engagement with the spider 57. FIG. 11 shows the sensor apparatus 285 in a non-deformed state (e.g., when no force is applied to the pedal 45). With reference to FIGS. 12 and 16, the first portion 395 of the engagement member 390 engages and acts upon the sensor apparatus 285 when a force is applied to the pedal 45 to cause rotation of the spider 57 (in the direction indicated by arrow 420 in FIG. 16) and thus the sprocket assembly 55. Generally, a substantial portion of the force transferred from the pedal 45 to the spider 57 is transferred directly through the sensor apparatus 285. Stated another way, the first insert 345 is indirectly acted upon by the pedal 45 (i.e., via the pedal spindle 50 and the crank arm 40) in response to pressure applied to the pedal 45, and transfers most, if not all, of the force directly to the central portion 335 through the sensor apparatus 285.

In particular, the engagement member 390 rotates into engagement with the sensor apparatus 285, and the force (indicated by arrow 425 in FIG. 12) of the first portion 395 acting on the sensor apparatus 285 deforms the cap plate 305 (i.e., moves at least a portion of the cap plate 305 relative to the shell 295) and rotates the spider 57. Deformation of the cap plate 205 moves the second sensor element 315 toward the first sensor element 310 a small amount, and the resulting change in the size of the gap 330 is detected by the sensor apparatus 285 and is used to determine the corresponding vector force 250 being applied to the pedal 45. Generally, the size of the gap 330 will vary depending on the magnitude of the force acting on the sensor apparatus 285. When the force acting on the pedal 45 is removed, the sensor apparatus 285 returns to the non-deformed state.

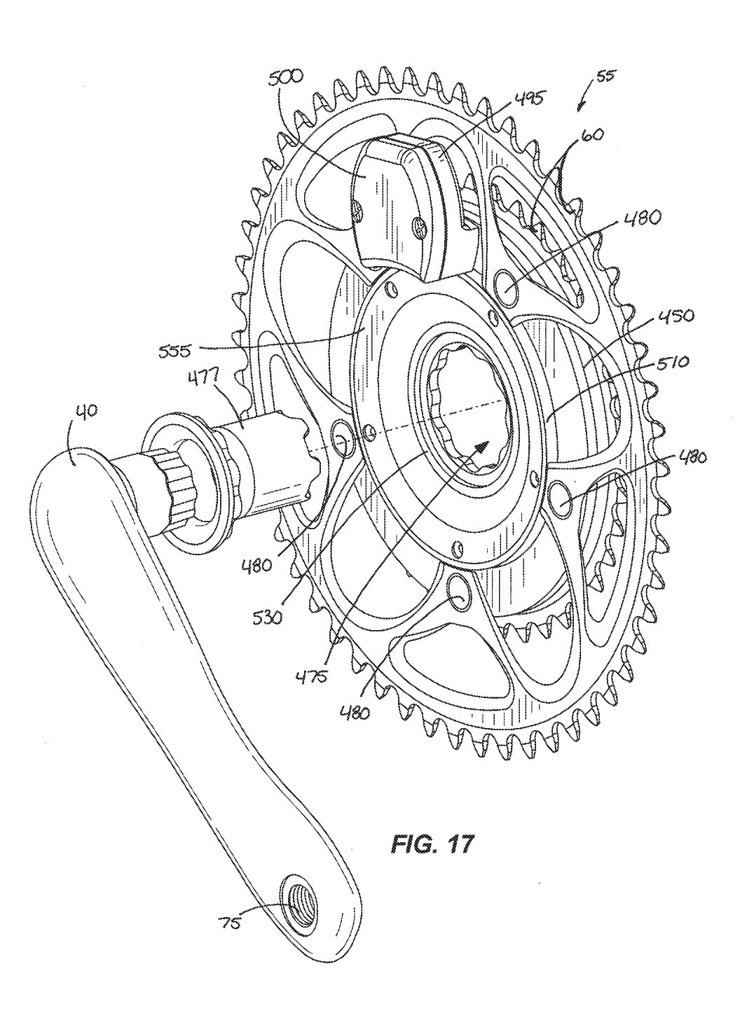

FIGS. 17-20 illustrate another spider 450 that can be positioned on the bicycle 10 to support the sensor apparatus 285 in lieu of the spider 57. The front sprocket assembly 55 is coupled to the crankset 35 by the spider 450 and include two chain rings 60 that couple to the chain 65.

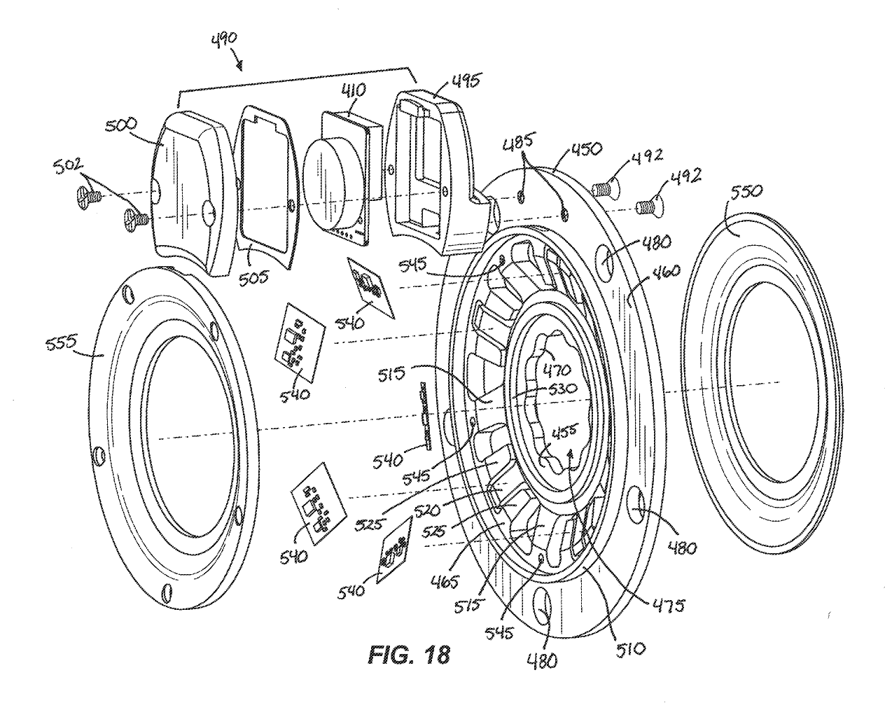

With reference to FIGS. 17 and 18, the spider 450 is disc-shaped and includes a central body or central portion 455, an outer annular region or portion 460, and an inner annular region or portion 465 disposed between and joined with the central portion 455 and the outer annular region 460. As illustrated in FIG. 17, a spindle portion 470 is disposed adjacent a periphery of an aperture 475 at the center of the central portion 455, and is sized and shaped to fit onto a spindle 477 of the crankset 35 that is coupled to the crank arms 40. With reference to FIGS. 17, 19, and 20, when force is applied to the pedal 45 to drive the bicycle 10 in a forward direction, the force transfers through the crank arm 40 and the spindle 477, which rotates the spider 450 in the direction indicated by arrow 478.

FIGS. 18-20 show that the outer annular portion 460 has five chain ring mounting holes 480 that are circumferentially spaced apart from each other along the outer periphery of the spider 450 and that support the chain rings 60. The outer annular portion 460 also has two additional mounting holes 485 disposed between two adjacent chain ring mounting holes 480 to support a housing 490 for the electronic module 410. The housing 490 is detachably coupled to the outer annular portion 460 via fasteners 492 that couple to the mounting holes 485. As illustrated, the housing 490 has a base 495 supporting the electronic module and a cover 500 that is attached to the base 495 using fasteners 502. A gasket 505 is positioned between the base 495 and the cover 500 to inhibit infiltration of dirt, water, and other debris into the housing 490.

An annular rib 510 extends around the spider 450 and is disposed between the outer and inner annular portions 460, 465. Stated another way, the annular rib 510 defines the inner periphery of the outer annular portion 460 and defines an outer periphery of the inner annular portion 465. The annular rib 510 acts as a stiffener for the spider 450. As illustrated in FIG. 17, the annular rib 510 has an axial height so that the annular rib 510 is substantially flush with the large chain ring 60.

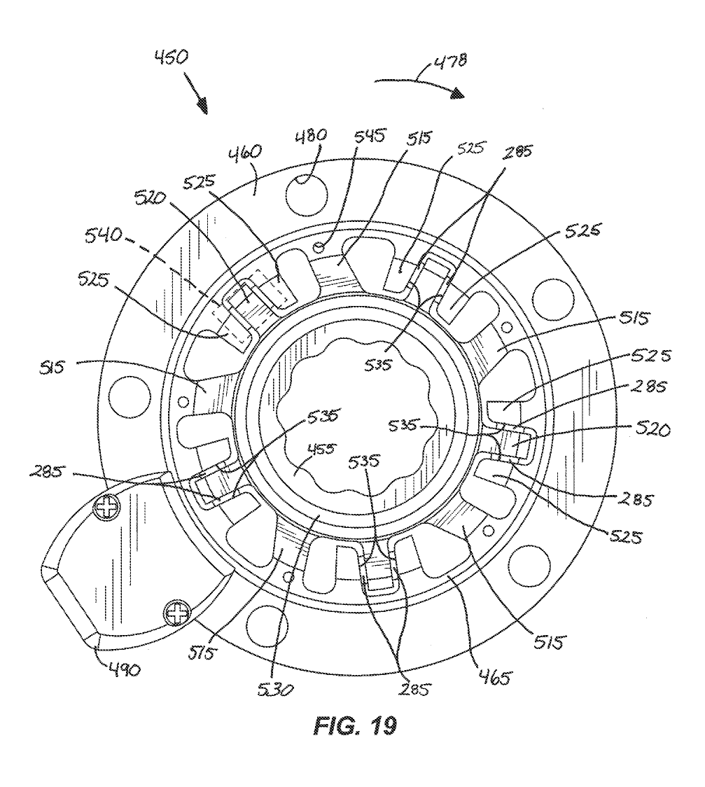

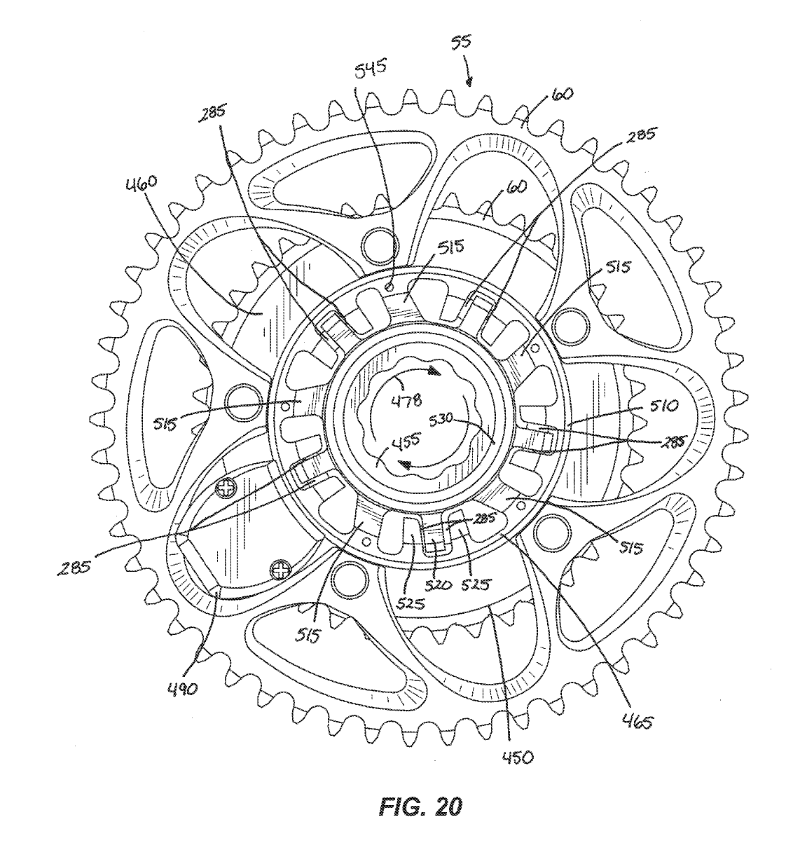

Referring to FIGS. 18-20, the inner annular portion 465 has arms 515 extending between and connecting the central portion 455 and the outer annular portion 460, and first and second fingers 520, 525 disposed between adjacent arms 515. As illustrated in FIGS. 19 and 20, the front edges of the arms 515 (relative to the direction of rotation 478 of the spider 450) are obliquely angled relative to radial axes of the spider 45. In other words, the arms 515 angle rearward relative to the direction of rotation 478 of the spider 450 from adjacent an annular ridge 530 surrounding the central portion 455 to the outer periphery of the inner annular portion 465. As illustrated, the rear edges of the arms 515 are substantially aligned with radial axes of the spider 450. In other constructions, the front edge of the arms 515 can be parallel to a radial axis, or angled forward relative to the direction of rotation 527. Also, the rear edges of the arms 515 can be obliquely angled forward or rearward relative to radial axes of the spider 450. Although five arms 515 are shown in FIGS. 18-20, fewer or more than five arms 515 can be provided on the spider 450 to transfer motive force to the chain rings 60.

The first fingers 520 extend outwardly from the central portion 455 adjacent an annular ridge 530 defining the outer limit or periphery of the central portion 455. As illustrated, the first fingers 520 extend toward the outer annular portion 460 without being connected to the outer annular portion 460. Stated another way, each first finger 520 defines a peninsula with a free end so that the first fingers 520 move in response to flexing of the arms 515. FIGS. 19 and 20 illustrate that each first finger 520 is disposed between and is annularly equidistant from two adjacent arms 515, although the first fingers 520 can be located anywhere between adjacent arms 515. Also, the illustrated spider 450 includes five first fingers 520, although fewer or more first fingers 520 can be provided.

The second fingers 525 extend inwardly from the annular rib 510 toward the central portion 455 without being directly connected to the annular ridge 530 or the central portion 455. Each second finger 525 defines a peninsula with a free end such that the first and second fingers 520, 525 are separated from each other by a narrow channel 535 (e.g., approximately 1-4 millimeters in width). As discussed in detail below, the channel 535 permits movement of the first fingers 520 relative to the second fingers 525 in response to flexing of the arms 515. FIGS. 19 and 20 show that each first finger 520 is positioned between two second fingers 525 (i.e., the two second fingers 525 surround the first finger 520), which cooperatively define a set of fingers 520, 525 that interact with each other and the sensor apparatus 285 to determine a vector force applied to the pedals 45. While the illustrated spider 450 includes ten second fingers 525, fewer or more second fingers 525 can be provided (e.g., one second finger 525 associated with one first finger 520 in each set). Each set of fingers 520, 525 is disposed annularly equidistant from adjacent sets of fingers, although other spacing arrangements are possible.

When pressure or force is applied to the pedal 45 to move the bicycle 10 forward, the spider 450 rotates in the direction of arrow 478. The illustrated arms 515 are formed (e.g., machined) to have a predetermined annular width or thickness (e.g., 6-13 millimeters) so that the arms 515 flex or yield in response to a force applied to the pedal 45. That is, the arms 515 are relatively narrow as illustrated in FIGS. 19 and 20 and define flex points of the spider 450. In the illustrated construction, the arms have a predetermined thickness of approximately 8 millimeters. Due to the connection of the first fingers 520 to the central portion 455 without a direct connection to the outer annular portion 460, and the direct connection of the second fingers 525 to the outer annular portion 460 without a direct connection to the central portion 455, and the channel 535 between the first and second fingers 520, 525, the first fingers 520 move slightly relative to the second fingers 525 in response to movement (flex) of the arms 515 caused by the force applied to the pedal 45.

As shown in FIGS. 18-20, ten sensor apparatus 285 are disposed in the spider 450 within the channels 535 to detect the force applied by the rider to the pedal 45. Each sensor apparatus 285 is bonded to the first and second fingers 520, 525 within the channel 535 and moves in response to relative movement between the fingers 520, 525. In other constructions, fewer or more than ten sensor apparatus 285 can be coupled (e.g., bonded) to the spider 450, depending in part on the quantity of first fingers 520 that are provided. For example, the spider 450 can include two sensor apparatus 285 (e.g., positioned in the two channels 535 of one set of fingers 520, 525, or positioned in one channel 535 of one set of fingers 520, 525 and in another channel 535 of another set of fingers 520, 525), leaving the remaining channels 535 without sensor apparatus 285. As few as one sensor apparatus 285 can be used, although a higher quantity of sensor apparatus 285 is preferable to determine the vector force applied to the pedals 45. Also, each sensor apparatus 285 has a width (along the axial direction of the spider 450) that is approximately equal to the thickness of the arms 515 so the sensor apparatus 285 does not protrude axially relative to the first and second fingers 520, 525.

The sensor elements 310, 315 in the sensor apparatus 285 are in electrical communication with a sensor board 540 (e.g., a printed circuit board) via electrical connections (not shown) that extend from the housing 290 to the sensor board 540 through a portion of the channels 535 adjacent the free ends of the first fingers 520. The sensor board 540 for each sensor apparatus 285 is coupled (e.g., adhered or bonded) to the set of fingers 520, 525 to which the sensor apparatus 285 is associated. As illustrated, the sensor boards 540 are bonded to one, some, or all of the fingers 520, 525 on the interior side of the spider 450. In some constructions, a sensor board 540 cover (not shown) can be placed over each sensor board 540.

With reference to FIGS. 17-20, each arm 515 has a hole 545 that receives a fastener (not shown) to attach an inner plate 550 and an outer plate 555 to the spider 450. The inner plate 550 overlays an interior side of the inner annular portion 465. The outer plate 555 overlays an exterior side of the inner annular portion 465 and covers the arms 515, the first and second fingers 520, 525, and the sensor apparatus 285. The inner and outer plate 555s enclose the inner annular portion 465 on both sides of the spider 450 to limit dirt, water, and other debris from infiltrating the inner annular portion 465 on which the sensor apparatus 285 are supported.

Each sensor apparatus 285 is positioned between the first and second fingers 520, 525 so that the first and second sensor elements 310, 315 are responsive to movement of the first finger 520 relative to the second finger 525. When a force is applied to the pedal 45 to cause rotation of the spider 450 (in the direction indicated by arrow 478 in FIG. 20), a substantial portion of the force acting to the spider 450 is transferred to the sprocket assembly 55 directly through the flexible arms 515. Due to the flex of the arms 515, a relatively small portion of the force acting on the spider 450 is transferred to the sprocket assembly 55 through the first finger 520, the sensor apparatus, and the second finger 525 in each set of fingers 520, 525. Stated another way, the arm 515 is indirectly acted upon by the pedal 45 (i.e., via the pedal spindle 50 and the crank arm 515 40) in response to pressure applied to the pedal 45, and transfers most of the force directly to the chain rings 60. The first fingers 520 are indirectly acted upon by the force on the pedal 45 due to flexing of the arms 515 and transfer most, if not all, of the remaining force to the chain rings 60 through the sensor apparatus 285 and the second fingers 525.

As discussed above, the relatively thin arms 515 flex slightly in response to torque acting on the spider 450 resulting from a force applied to the pedals 45 to move the bicycle 10 forward. With reference to FIGS. 19 and 20, the flex of the arms 515 is substantially or completely taken up by and causes movement of the first finger 520 in each set of fingers 520, 525 toward the second finger 525 on the forward side of the first finger 520 and away from the second finger 525 on the rearward side of the first finger 520 (relative to the direction of rotation 478). As illustrated in FIG. 20, movement of the first finger 520 relative to the surrounding second fingers 525 acts upon and compresses the sensor apparatus 285 on the forward side, and acts upon and expands the sensor apparatus 285 on the rearward side.

The size of the gap 330 will vary depending on the magnitude of the force acting on the sensor apparatus 285. When no force acts on the pedal 45, the sensor apparatus 285 is in the non-deformed state. As described with regard to FIGS. 11-16, the force of the first finger 520 acting on the sensor apparatus 285 on both sides of the first finger 520 deforms the cap plate 305 (i.e., moves at least a portion of the cap plate 305 relative to the shell 295). Deformation of the cap plate 205 of the sensor apparatus 285 on the forward side moves the second sensor element 315 toward the first sensor element 310 a small amount. Likewise, deformation of the cap plate 205 of the sensor apparatus 285 on the rearward side moves the second sensor element 315 away the first sensor element 310 a small amount. The resulting change in the size of each gap 330 is detected by the sensor apparatus 285 and is used to determine the corresponding vector force 250 being applied to the pedal 45. That is, the force 250 applied to the pedal 45 correlates to the dynamic size of the gap 330, or the change in the size of the gap 330. In some constructions, the electronic module 410 can correlate the size of the gap 330 to the force 250 using a look-up table.

By providing sensor apparatus 285 on opposite sides of the first finger 520 and responsive to a force acting on the pedal 45, the electronic module 410 can determine the positive and negative forces around the crankset 35. In particular, compression of the sensor apparatus 285, which decreases the size of the gap 330, is detected by the electronic module 410 as a positive or increasing force acting on the sensor apparatus 285. Expansion of the sensor apparatus 285, which increases the size of the gap 330, is detected by the electronic module 410 as a negative or decreasing force acting on the sensor apparatus 285. The electronic module 410 evaluates these positive and negative forces (corresponding to the sizes of the gaps 330) around the spider 450 to determine the force being applied to the pedal 45. In some constructions, the electronic module 410 can correlate the positive and negative forces to determine whether the sensor apparatus 285 are working properly.

The sensor apparatus 285, in some contexts, is a simplified version of the sensor apparatus 85. Placement of the sensor apparatus 285 remote from the pedals 45 (e.g., in the bottom bracket 37, the spider 57, the rear hub 72, or in other locations on the bicycle 10), where the corresponding bicycle component (e.g., insert 345) is acted upon indirectly by the pedal 45, also provides accurate measurements of the resultant force vector 250 stemming from the force applied to the pedal 45. Remotely locating the sensor apparatus 285 relative to the pedals 45 means that the pedal force indirectly acts on the sensor elements 320, 325 (e.g., through the crank arm 40 and the spider 57). As desired, additional sensors (e.g., an accelerometer, etc.) can be used in conjunction with the sensor apparatus 285 to provide more detailed information (e.g., power, etc.) regarding pressure being applied to the pedals 45. These additional sensors can be incorporated into the electronic module 410 or separately coupled to the bicycle 10.

Various features and advantages of the invention are set forth in the following claims.

1. A bicycle comprising:
a frame having a bottom bracket;
a crankset attached to the bottom bracket and including a sprocket assembly and a spider, the spider having a flexible arm;
a pedal coupled to the crankset and operable to propel the bicycle in response to a force acting on the pedal; and
sensor apparatus including a sensor element positioned to sense a force transferred from the pedal to the sprocket assembly and indicative of the force acting on the pedal.

2. The bicycle of claim 1, wherein the spider includes a central portion operatively coupled to the pedal and an outer portion coupled to the central portion via the flexible arm, and wherein the sensor element is responsive to yielding of the flexible arm.

3. The bicycle of claim 2, wherein the spider further includes a first spider element coupled to the central portion and responsive to the force acting on the pedal, and a second spider element separated from the first spider element by a channel, and wherein the sensor apparatus coupled to the spider within the channel and responsive to movement of the first spider element relative to the second spider element.

4. The bicycle of claim 3, wherein the spider includes two second spider elements surrounding the first spider element, one of the second spider elements separated from the first spider element by a first channel and the other of the second spider elements separated from the first spider element by a second channel, and wherein the bicycle includes first sensor apparatus disposed in the first channel to detect a positive force component of the force applied to the pedal, and a second sensor apparatus disposed in the second channel to detect a negative force component of the force applied to the pedal.

5. The bicycle of claim 1, wherein the flexible arm has an annular width of less than 10 millimeters.

6. The bicycle of claim 5, wherein the spider includes a plurality of flexible arms.

7. The bicycle of claim 1, wherein a substantial portion of the force acting on the pedal is transferred directly through the flexible arm.

8. The bicycle of claim 1, wherein the sensor element is a first sensor element, the sensor apparatus further including a second sensor element, wherein the first sensor element and the second sensor element are enclosed by a housing, and wherein the second sensor element is spaced apart from the first sensor element and is movable relative to the first sensor element in response to the force applied to the pedal.

9. The bicycle of claim 8, further comprising a detector in communication with the first sensor element and the second sensor element to detect at least one of a change in distance and a change in volume between the first and second sensor elements, the change in distance or volume indicative of the force applied to the pedal.

10. A bicycle comprising:
a frame having a bottom bracket;
a crankset attached to the bottom bracket and including a sprocket assembly and a spider, the spider having a first spider element and a second spider element, the first spider element movable relative to the second spider element;
a pedal coupled to the crankset and operable to propel the bicycle in response to a force acting on the pedal; and
sensor apparatus including a sensor element disposed between the first spider element and second spider element to sense a force transferred from the pedal to the sprocket assembly and indicative of the force acting on the pedal.

11. The bicycle of claim 10, wherein the spider includes a central portion operatively coupled to the pedal, and wherein the first spider element is coupled to the central portion and responsive to the force acting on the pedal.

12. The bicycle of claim 10, wherein a portion of the force acting on the pedal is transferred to the sprocket assembly through the first spider element and the second spider element.

13. The bicycle of claim 12, wherein the spider further includes a flexible arm, wherein a substantial portion of the fore acting on the pedal is transferred through the flexible arm.

14. The bicycle of claim 13, wherein the first spider element is movable in response to yielding of the flexible arm.

15. The bicycle of claim 12, wherein the sensor element is a first sensor element, the sensor apparatus further including a second sensor element, wherein the first sensor element and the second sensor element are enclosed by a housing, and wherein the second sensor element is spaced apart from the first sensor element and is movable relative to the first sensor element in response to the force applied to the pedal.

16. The bicycle of claim 15, further comprising a detector in communication with the first sensor element and the second sensor element to detect at least one of a change in distance and a change in volume between the first and second sensor elements, the change in distance or volume indicative of the force applied to the pedal.

17. A crankset for a bicycle including a pedal, the crankset comprising:
a sprocket assembly;
a spider including a central portion rotatable in response to a force applied to the pedal, and an arm disposed between the sprocket assembly and the central portion and flexible in response to rotation of the central portion; and
sensor apparatus including a sensor element positioned to sense movement of the flexible arm.

18. The bicycle of claim 17, wherein the spider further includes a first spider element coupled to the central portion and movable in response to yielding of the flexible arm, and a second spider element separated from the first spider element by a channel, and wherein the sensor apparatus is disposed in the channel and responsive to movement of the first spider element relative to the second spider element.

19. The bicycle of claim 18, wherein the spider includes two second spider elements surrounding the first spider element, one of the second spider elements separated from the first spider element by a first channel and the other of the second spider elements separated from the first spider element by a second channel, and wherein the bicycle includes first sensor apparatus disposed in the first channel to detect a positive force component of the force applied to the pedal, and a second sensor apparatus disposed in the second channel to detect a negative force component of the force applied to the pedal.

20. The bicycle of claim 17, wherein a substantial portion of the force acting on the pedal is transferred directly through the flexible arm.
